(19) United States
(12) Patent Application Publication
(10) Pub. No.: US 2009/0054814 A1
(43) Pub. Date: Feb. 26, 2009

(54) POSTURE MONITORING DEVICE AND METHOD OF USE THEREOF

(76) Inventors: ELMA O. SCHNAPP, MEMPHIS, TN (US); MOACIR SCHNAPP, MEMPHIS, TN (US); ERIC C. SCHNAPP, MEMPHIS, TN (US); WILLIAM D. SCHNAPP, MEMPHIS, TN (US); DENISE E. SCHNAPP, MEMPHIS, TN (US)

Correspondence Address: WYATT, TARRANT & COMBS, LLP, 1715 AARON BRENNER DRIVE, SUITE 800, MEMPHIS, TN, 38120-4367, US

(21) Appl. No.: 12/202,732

(22) Filed: Sep. 2, 2008

Related U.S. Application Data
(63) Continuation-in-part of application No. 11/066,505, filed on Feb. 25, 2005.

(51) Int. Cl. A61B 5/103

(52) U.S. Cl. 600/595

(57) ABSTRACT

The present claimed subject matter teaches a posture monitoring device. The device includes a tilt sensor, an alarm, and a recording device to track changes in posture. The device has been designed to overcome the currently existing problems associated with other devices that detect poor postures. The device also includes the ability to easily modify the target posture angle and the amount of time during which poor posture is detected prior to activation of the alarm.

### CROSS-REFERENCE TO RELATED APPLICATIONS

This application is a continuation-in-part application of co-pending U.S. application Ser. No. 11/066,505, filed Feb. 25, 2005 and entitled Posture Monitoring Device and Method of Use Thereof, which application is hereby incorporated by reference.

### STATEMENT REGARDING FEDERALLY SPONSORED RESEARCH OR DEVELOPMENT

Not applicable

### REFERENCE TO A “MICROFICHE APPENDIX”

Not applicable

### FIELD OF THE INVENTION

The present claimed subject matter relates generally to the field of posture management. Specifically, it relates to a device which identifies incorrect posture and signals for the correction of a user's posture.

### BACKGROUND OF THE INVENTION

The health and comfort of humans is enhanced by maintaining proper posture while standing, sitting, etc. Medical reports have clearly shown that maintaining correct posture results in fewer injuries. Additionally, with regard to elderly women, correct posture during the medical therapy for osteoporosis is a necessity. As females age, they may be susceptible to calcium loss, resulting in osteoporosis. Although medications are available to help restore the calcium and increase bone strength, unless the patient's posture is corrected during therapy, it is possible that the rounded shoulders and slumping posture, often associated with osteoporosis, will remain after treatment. Further, proper posture prevents curvature of the spine, such as kyphosis and scoliosis, even for individuals not subject to osteoporosis treatment.

Other attempts have been made to provide a device which alerts a user to correct their posture. However, each of the previous attempts is flawed with regard to accurately assessing the angle of displacement, the ability to effectively alert a user in a discrete and effective manner, and/or the ease of use. For example, previous attempts have often used either belts or some form of elastic band to affix the monitoring device to a user's torso. This placement, however, fails to achieve a high enough position on the user's body necessary to obtain the most accurate measurement. Instead measurements are frequently affected by the simple act of breathing or the measured displacement may be so minimal at a lower position that it may be imperceptible to the apparatus.

Another example of inferior prior designs involves the use of single or double axis sensors for measuring the angular displacement of a user. The use of these more primitive sensors necessitates the placement of the sensor in a position perpendicular to the torso and subsequently results in a larger overall device. By extending from the torso of the user, the device is also more easily affected by simple movements of the body including basic actions such as walking.

Numerous previous attempts have, therefore, resulted in cumbersome devices which are limited in their functionality because of their design and are routinely difficult to use and require the assistance of a therapist or other health care provider. Thus, it is an object of the current invention to provide a posture alert device which may be worn inconspicuously and which effectively measures and alerts a user when the need to correct posture arises.

### SUMMARY OF THE INVENTION

The proposed invention discloses an improved posture alert device used to alert a wearer of their poor posture. The device provides the advantage of more accurately measuring the angle of a wearer's posture while simultaneously minimizing the number of false notifications received by the user for alleged poor posture. Additionally, as the user transitions through various positions, for example from a standing position to a seated position, the device allows the user to easily reset it so that the user's posture is accurately monitored. The device also maintains a record of the wearer's posture swings so that such information may subsequently be analyzed.

In certain embodiments, the device includes a circular housing which is integral to the device's ability to accurately interpret angle measurements while not restricting the user's movement or comfort. Traditional posture alert devices utilize some form of thoracic strap to attach the device to a user's body. This strap is usually located at the highest position possible with a strap, under a user's armpits, which may restrict the user's movement and produce false alerts. The current invention's small size and circular shape both help to alleviate this problem by allowing for a variety of attachment methods of the device, none of which involves a thoracic strap or a limitation of mobility. In fact, certain embodiments of the device may have a clip, attachment loop, or adhesive to facilitate attachment to the wearer. Regardless of which method of attachment is utilized, the device is designed to be smaller and lighter than previous devices and can, therefore, rest comfortably on the manubrium region of the sternum. By resting on this upper region of the sternum the current invention is able to more accurately sense deviations in the posture of an individual.

A monitoring device's correct placement on a body is key to the ability of the device to accurately monitor posture. For example, a device to measure posture can be greatly affected by the simple act of breathing. When a user takes a breath, the user's chest expands and depending on the placement of the monitoring device, that displacement may vary. Placement of the device on the upper region of the chest, however, minimizes this effect. Furthermore, particularly in the case of women, a thoracic strap may pose particular concern. While one may think that a typical woman's brassiere may use elastic and therefore the use of such material is ok, this may not be the case. The elastic used in a woman's brassiere is used to support the area around the breasts and not press down on them. The thoracic strap, however, works to press down on the upper portion of a woman's breasts and concentrates that pressure on this sensitive area. This pressure may actually cause pain in a woman, which would subsequently prohibit her from moving and acting as she normally would.

The unique positioning of the device is further aided by other characteristics of its design. Although the device's back is relatively flat in design, it is angled slightly toward the middle of the device. This angle is crucial to giving the device greater stability. This angle on the back of the device allows it to fit right into the cleavage of a user, rather than resting on top of it. In fact, the angled back of the device helps to create a wedge for the device which assists in maintaining its placement. This not only allows the user to place the monitor in the best position to measure posture (a substantial improvement over the prior art) but improves the functionality of the device as well.

By allowing the device to be placed closer to the manubrium region of the sternum, the efficacy of the discrete vibration alert system will be improved as well. As the device monitors a user's posture it is necessary to provide alerts to the user when the device senses that the user has fallen outside of a set range of motion. In order to notify the user, the device employs a variety of notification methods including audible notification, visual notification, and tactile notification. The tactile notification employs a vibration motor within the device to produce a vibration similar to that used in cellular phones or other small, portable electronic devices. By utilizing the device's unique wedge shape and close placement to the upper portion of the sternum on the chest, a gentler vibration may be utilized by the device and easily perceived by the user as it is transmitted via bone conduction.

Traditional vibration devices required a more powerful vibration mechanism in order to traverse a larger gap between the user and the device placement, which also helped to diminish the device's discreet notification mechanism. In fact, several prior art references utilize a device placement on top of clothing. This placement, removed from direct contact with the user's body and more importantly the user's bone, allows for the vibration to be largely absorbed by the excess skin and clothing, leaving little to be felt by the user. Many alerts may go unnoticed with such remote placement, thereby defeating the purpose of the device. Furthermore, even those devices which sit on a users chest fail to provide the effective level of notification seen in the current invention. Devices which utilize a thoracic strap and are meant to sit on the user's chest must traverse an area which is usually padded with skin and thick tissue such as found on the breasts. Any vibration alert must traverse this padded region and the result is a largely decreased intensity perceived by the user and the need for a more powerful vibration mechanism.

A further distinction of the current invention involves the improved method utilized to set the threshold displacement value for a user. This improved method is aided by both the physical design of the device and the improved software which is part of the current invention. With regard to the physical design, while many prior inventions utilize a series of buttons on the device in order to set the relevant threshold limits, none effectively takes into account and compensates for the inherent movement of the device due to the button press. When a user presses a button on the device to set a threshold angle, this action pushes the entire device up against the chest and can result in the recording of an incorrect angle by the device. The physical design of the current invention, however, minimizes any displacement of the device by keeping the device at a constant position near the upper sternum which minimizes any possible movement of the device. Additionally, the contoured design of the back of the device allows for a greater surface area to remain in contact with the user, which helps to reduce the potential movement of the device.

To further improve the accuracy of the device when setting the threshold angle, the software on the current invention has been designed with several improvements over the prior art. First, while the prior art requires the user to set both a forward and backwards angle, the current invention simply requires the user to set a forward angle threshold in order to effectively monitor posture. Additionally, this forward angle threshold can be optimally set while the user is wearing the device, without the need of any external connection to a computer. The location of the device on a user's body is critical when measuring the user's posture. Each user has a different body including differing features in the chest, back, and spine which prevent any universal parameters from working for all users. Instead, the device must be programmed for each individual user's unique characteristics.

The improved software also includes an innovative algorithm used to interpret the abundance of information sensed by the three-axis inclinometer within the device. Because the inclinometer can register movement in the X, Y, and Z axes, any movement in any direction can be registered and must be filtered appropriately. For example, the simple act of walking may be interpreted by the device as a change in angle and motions such as this must therefore be filtered by the device. The novel algorithm developed helps to evaluate the input received from the inclinometer and filter the results to help prevent false notifications for the user.

The software further represents a novel method of battery conservation not found in the prior art. The first method of conservation involves the use of a unique sleep/wake cycle to measure a user's posture. The device “sleeps” in a low power consumption state and “wakes up” at predetermined intervals to measure the user's current angle of displacement. If the device senses the user's displacement has not exceeded the set threshold limit, it returns to sleep and waits another pre-determined cycle to repeat the process. If, however, the device detects that the user's angle of displacement has exceeded the threshold limit set by the user, the device vibrates once and enters a shortened sleep cycle. If following that second sleep cycle it still measures the user's angle of displacement above the threshold limit, it vibrates twice to notify the user and returns to the sleep cycle awaiting the next predetermined wake up time. If on the third measurement the user's displacement is still outside the set threshold level, the device will intelligently assume the user desires to remain in this position and the device will enter an extended sleep cycle to conserve power. After each subsequent wake cycle in which the device determines the user is still outside the threshold limits it will once again enter an extended sleep cycle in an effort to conserve power. Once the device senses that the user has returned to a position within threshold limits, it will resume its normal shortened sleep/wake cycle.

A further power saving design of the device involves the interplay between the device's physical design and the novel software designed for the device. When a user no longer wishes to wear the device, it may be set down on a table, desk, etc. and the shape of the device will cause it to naturally lay down flat. When the device wakes from its sleep cycle, the software will register the device's position as being well beyond the threshold angle. The software will then interpret this extreme angle and instruct the device to “sleep” with no predetermined time to wake up. When the user wishes to again use the device, they will resume wearing the device and press the set button on the device to once again set the reference angle.

An additional factor necessitating this unique programming is the location at which the user places the device on the body. While a specific threshold angle may equate to proper posture at one location on a user's body, merely moving the device down one inch on the chest of the same user may represent improper posture. Therefore, the device must be easily re-programmed each and every time it is worn. In order to assist with setting this angle accurately, the device uses an innovative method of waiting a fraction of a second after the user presses the button before recording the threshold angle. This delay allows the user time to finish the button press while still maintaining the desired threshold angle and allows the device time to return to its natural position on the user's chest. A second advantage associated with this delay, involves the vibration feedback the user receives when setting the threshold angle. Once the user presses the button to record the threshold angle, the device vibrates to notify the user of a successful action. This vibration is carried through the entire circuit board and may affect the angle the inclinometer senses at the time the threshold angle is set. For this reason, the fractional delay in recording said angle allows for the effects of the vibration to be minimized.

A further improvement of the current invention over the prior art involves the use of a single three-axis inclinometer in place of the prior arts maximum two-axis inclinometers. Therefore, the current invention's ability to measure displacement of a third angle represent a novel improvement by allowing the device to be smaller and more compact. Traditional two-axis devices require the inclinometer to be placed perpendicular to the user and therefore require the posture monitoring device to be fairly thick in size. This thicker size results in a device of greater weight which may affect the accuracy of the device. As a user walks, the body moves in an up and down motion and everything attached to the body will follow this motion. A bulkier, heavier device will be more affected by this motion than a smaller, lighter device. As the user takes a step, the entire body first rises up and any device attached to the user's chest will similarly rise with it. The user's body must then fall on one foot in that stride and although the user's body has then begun its descent, the device's inertia will allow it to continue to rise until affected by an outside force. The outside force necessary is dependent upon the mass of the object so a heavier device would require a higher force before it would change direction and proceed downward with the user. The current invention has a novel design which is smaller and lighter than any previous design. This novelty allows the device to easily mimic the movements of the user and allows the device to more accurately measure a user's movement.

In still other embodiments, the device further includes a timing sensor and a monitoring circuit for monitoring the maximum swings of the spine angle. Other embodiments may include a recorder, also called a memory storage device, for recording the maximum swings of the spine angle. Still other embodiments include a memory storage device for recording and playing back the recorded angles. In certain embodiments, the angle detecting sensor may be digital. In other embodiments the housing may have a length of up to 1.35 inches and a width of up to 1.10 inches.

Accordingly, one object of the present invention is to provide an improved device for conveniently and accurately monitoring the posture of a user.

Another object of the present invention is to provide a device that comfortably sits in a high enough position on the user's sternum and allows for increased accuracy and minimal false alarms.

Still another object of the present invention is to provide a device that provides effective and discrete notification to the user.

Another object of the present invention is to provide a software for the device which minimizes the power utilized by the device and maximizes its efficiency.

Yet another object of the present invention is to provide a small device which may be quickly and conveniently removed when a user wants to stop monitoring his or her posture.

### BRIEF DESCRIPTION OF THE DRAWINGS

For a further understanding of the nature, objects, and advantages of the present invention, reference should be had to the following detailed description, read in conjunction with the following drawings, wherein like reference numerals denote like elements and wherein:

FIG. 1A shows a cross section of an elevated side view of a first embodiment of the present invention.

FIG. 1B is a front view of a first embodiment of the posture monitoring system showing the multi-function button in the center.

FIG. 1C is a perspective view of the posture monitoring system, without the housing, showing the components attached to the circuit board.

FIG. 2 is a side view of a user in an upright position.

FIG. 3 is a front view of a second embodiment of the present invention with dashed lines showing the wall thickness of the housing and phantom lines showing the positioning of the alarm, second circuit board, and tilt sensor on the underside of the first circuit board.

FIG. 4 is a flow chart showing the steps of using the present invention.

### DETAILED DESCRIPTION OF THE INVENTION

The present invention is a posture monitoring system 10, also called a posture alert device, which includes a housing 12, a timing sensor in the form of a microprocessor 42, a tilt sensor 16, also called an inclinometer, an alarm 18, and monitoring circuit hardware that includes a memory storage device 44. The posture monitoring system 10 provides an alert, or notice, to a user when the user's posture is in violation of the specific settings of the system 10. As further described herein, the system 10 may be set to for a specific tilt threshold, and the amount of time during which a user may be in violation of a specific tilt range.

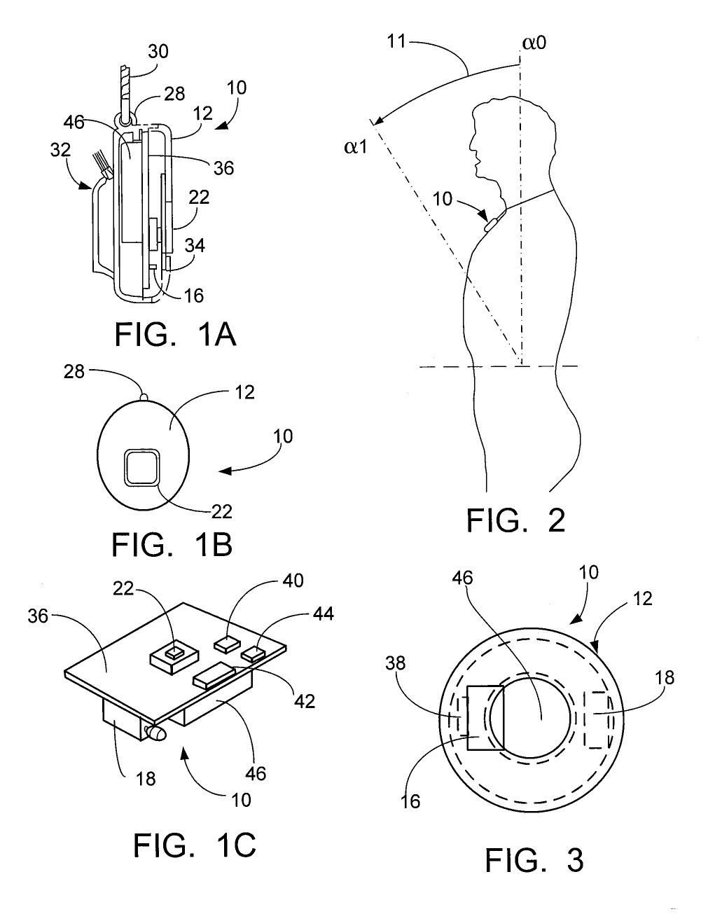

Shown in FIGS. 1A and 1B is a first embodiment of the present invention. FIG. 1B shows a front view of the invention having a cylindrical shape. FIG. 1A, which is a cross sectional view, shows the housing 12, the inclinometer 16, the multi-function button 22, the circuit board 36, the elastomeric membrane 34 covering the multi-function button 22, the clip 32, the battery 46, and the attachment loop 28 attached to a necklace 30. Shown in FIGS. 1A and 1B is the length of the system 10. In one embodiment, the value of the length may be 1.5 inches. In alternate embodiments, the length may be just 1 inch. Furthermore, in certain embodiments the width is 0.48 inches. Thus, the system 10 is contained within an extremely small structure. Furthermore the system 10 is unitary and does not result in the need to affix various structures to different parts of the user's body.

The system 10 may be constructed as disclosed herein. The housing 12 may be constructed of any rigid material, which is sufficient to provide a point of attachment for the other components of the invention disclosed herein. By way of illustration, and not limitation, examples of metal, plastic, rubber, glass, or ceramic material. In certain embodiments, the housing 12 wall may have a thickness of approximately 0.010 inches to approximately 0.070 inches, depending upon the material of construction.

In certain embodiments of the present invention, the system 10 is capable of storing data for evaluation purposes. Accordingly, in order to facilitate the transfer of data, it is necessary that the system include an RS-232, USB, Bluetooth, or some other adequate connection method and a form of rewritable Flash RAM Memory. For example, each time an alert occurs, the recorded data may include the time, date, and tilt angle which resulted in the violation. Accordingly, the recorded data may be stored as a comma delimited file so that it may be transferred via a USB data connection, or the like, into a spreadsheet, or the like. The recorded data may include the time stamp and the reference tilt angle 11. When the recorded data is displayed, on a monitor or in printed form, the data may be analyzed for medical diagnostic purposes. Alternatively, certain embodiments of the present invention may have wireless data transmission via Bluetooth or infrared transmitters or other appropriate wireless transmission. With regard to the power source, the invention may include a replaceable battery source or a rechargeable battery source. The manner of transferring data through the described avenues is well known in the art and is easily accomplished by one of ordinary skill in the art.

With regard to the inclinometer 16 of the present invention, the inclinometer measures an individual's movement in the x, y, and z axes. While it is not necessary to measure all three axes of movement, by doing so the device is able to perceive a much more complete picture of the user's tilt and rotation.

Having disclosed how to make the system 10, information is now provided with regard to using the system 10. A user may attach the device 10 using a variety of attachment methods. The first involves the use of a clip 32 to attach the device to a shirt or under garment. A second attachment method involves the use of the device 10 as a pendant on an attachment loop 28 worn around the neck. Finally, the device 10 may also be attached directly to a user via the use of a medical grade adhesive backing attached directly to the device. Regardless of the attachment method utilized, the device 10 should be situated on the upper region of a users chest in or parallel to the manubrium region of the sternum. This placement allows for increased sensitivity of the device 10 while minimizing the instance of false alarm notifications. Additionally, this placement allows the user to set the notification alarm to vibrate so that the user is discretely notified when a posture violation occurs. In such a situation, the user easily feels the vibration as it is conducted via the sternum, with little perceptible sound. As further described below and illustrated in FIG. 2, the user sets the forward allowable posture threshold for monitoring and notification by the device. The user accomplishes this by leaning forward to the desired threshold angle al and presses the multi-function button 22 to establish this reference tilt threshold in the device 10. The angle measured by the inclinometer is then measured every 0.01 seconds, based upon the device's software and the chain of previous events. When the inclinometer 16 measures a tilt angle 11 greater than the preset posture tilt angle threshold 11, the user receives notification by way of an alarm mechanism 18. in certain embodiments, the alarm 18 may be signaled as a pulse of approximately one second of a vibrator motor. In certain embodiments, if, after a one minute period of exceeding the set threshold 11, the user's posture continues to be in violation, the vibrator motor will then again pulse for approximately one second. This will continue in one minute intervals for a total of two cycles until the user either corrects their posture or the device 10 assumes the user is ignoring the notifications and enters an extended sleep cycle.

In certain embodiments of the present invention, the device's 10 design, as further illustrated in FIG. 3, helps to facilitate power conservation by placing the device in a forced sleep mode when not being worn. If the device 10 senses placement which is less than 20 degrees or more than 120 degrees from horizontal it will enter a wait state of one minute in which it will re-evaluate its angle of placement. If upon that second reading, the angle of placement remains less than 20 degrees or more than 120 degrees from horizontal, the device 10 will assume the user is no longer wearing the device and will enter an extended sleep mode which requires the user to depress the multi-function button 22 to once again establish the reference tilt threshold of the device 10.

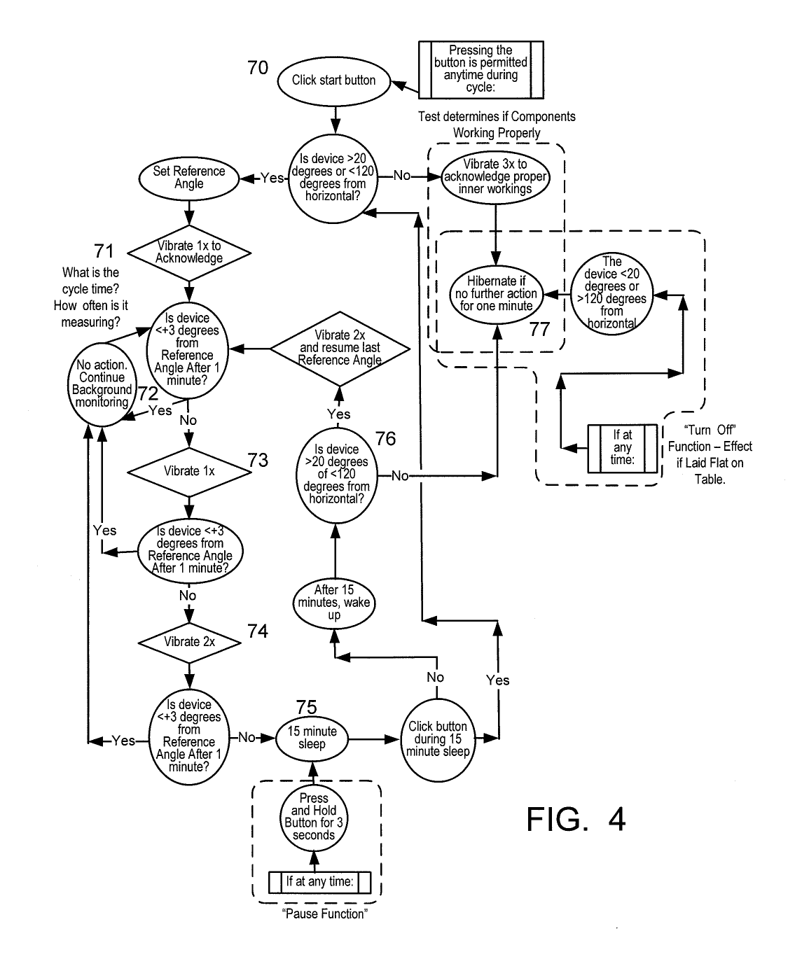

Referring specifically to FIG. 4, there is shown a flow chart providing the steps for utilizing an embodiment of the device 10 including establishing a reference tilt threshold angle 11 for the device 10 to use when monitoring a user's posture. A user presses the multi-function button 22 while tilted to a maximum threshold position in order to set that angle as the reference tilt threshold 11 to be monitored by the device 10. As illustrated by step 71, the device 10 vibrates once to notify the user that a reference angle has been set. Once a reference tilt angle 11 is stored in the device, step 72 illustrates the on-going monitoring performed by the device. The device monitors a user's position one hundred time a minute after the reference tilt angle 11 is set, the device determine if the user's current angle of posture has deviated no more than three degrees from the set reference tilt angle 11. So long as the deviation remains less than three degrees for a period of one minute or greater, the device 10 simply continues to monitor the user. If the device 10 detects a user's position is outside the set threshold for a period greater than one minute, it will vibrate once to alert the user and continue monitoring the user's position. If the threshold is exceeded for an additional minute, the device will vibrate twice and enter a sleep period in which it will cease to monitor the user's position for 15 minutes as illustrated in step 75. After this extended sleep cycle, the device will once again evaluate the user's posture and as illustrated in step 76, if the user's posture is within twenty (20) degrees and one-hundred twenty (120) degrees from horizontal, the device will vibrate twice to notify the user and will resume monitoring the original set reference tilt angle 11. If the device, however, is outside those limits, the device 10 will enter an extended “hibernate” mode as illustrated by step 77 in an effort to conserve battery power.

The embodiments shown and the procedures set forth above are intended to simply be illustrative and are not intended to limit the scope of the claimed subject matter, it being intended that all equivalents thereof be included in the scope of the appended claims.

What is claimed is:

1. A posture monitoring device, comprising:
a housing;
an angle detecting sensor;
an alarm attached to the angle detecting circuit; and
a monitoring circuit attached to the angle detecting circuit for monitoring the maximum deviations of the spine angle.

2. The device of claim 1, further comprising a recorder for recording the maximum deviations of the spine angle.

3. The device of claim 1, wherein the recorder further comprises a memory storage device recorder for recording and playing back the recorded angle measurements.

4. The device of claim 1, wherein the angle detecting circuit further comprises a digital angle detecting sensor.

5. The device of claim 1, wherein the alarm further comprises a light.

6. The device of claim 1, wherein the alarm further comprises a vibrator unit.

7. The device of claim 1, further comprising a switch attached to the housing, wherein the switch may be used for a plurality of functions.

8. A posture alert device for alerting a user of their posture, comprising:
a housing, wherein the housing is of a cylindrical shape;
a circuit board attached to the housing for allowing a reference angle to be set and monitoring said angle;
a tilt sensor attached to and operationally connected to the circuit board; and
a vibrator motor attached to and operationally connected to the circuit board.

9. The device of claim 8, further comprising a switch attached to the circuit board.

10. The device of claim 9, further comprising an attachment loop attached to the housing.

11. The device of claim 9, further comprising a clip attached to the housing.

12. The device of claim 9, further comprising a medical grade adhesive attached to the housing.

13. A method of using the posture alert device, comprising:
a method for setting an initial reference alert angle;
a method of continued monitoring of a user's posture angle as specified intervals;
a method for notifying a user when outside the reference angle;
a method for placing the posture alert device in a lower power storage state.

14. The method of claim 13, characterized in that said specified interval for continued monitoring is one (1) minute.
